(19) United States
(12) Patent Application Publication
(10) Pub. No.: US 2003/0228277 A1
(43) Pub. Date: Dec. 11, 2003

(54) Use of ROM production and release inhibitors to treat and prevent intraocular damage

(76) Inventor: Kurt R. Gehlsen, Encinitas, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/403,390

(22) Filed: Mar. 28, 2003

Related U.S. Application Data
(60) Provisional application No. 60/369,085, filed on Mar. 29, 2002.

(51) Int. Cl. A61K 38/20; A61K 31/4172; A61K 31/375; A61K 31/203

(52) U.S. Cl. 424/85.2; 424/94.4; 514/400; 514/458; 514/474; 514/559

(57) ABSTRACT

A method of treating or preventing intraocular damage caused by reactive oxygen metabolites is provided. The method includes identifying a subject presenting the symptoms of proliferative diabetic retinopathy; and administering to at least one eye of the subject a pharmaceutically acceptable solution containing an effective concentration of a compound effective to reduce the amount of ROM in an individual. The compounds effective to reduce the amount of ROM in an individual include histamine and histamine related compounds. The specific disease states characterized by intraocular damage caused by reactive oxygen metabolites include proliferative diabetic retinopathy, preproliferative diabetic retinopathy, proliferative retinopathy, age-related macular degeneration, retinitis pigmentosa, and macular holes. A pharmaceutical composition including a pharmaceutically acceptable ophthalmic solution containing an effective concentration of a compound effective to reduce the amount of ROM in an individual is likewise provided.

### RELATED APPLICATIONS

The present application claims priority to U.S. Provisional Application Ser. No. 60/369085 entitled USE OF ROM PRODUCTION AND RELEASE INHIBITORS TO TREAT AND PREVENT INTRAOCULAR DAMAGE, which was filed on Mar. 29, 2002. The entire contents of the aforementioned provisional application are hereby incorporated by reference in their entirety.

### FIELD OF THE INVENTION

Described herein are compositions and methods for treating intraocular damage caused by trauma, autoimmune disease, degenerative diseases and cellular release of reactive oxygen species or inflammatory cytokines. More specifically treatment of macular degeneration through the delivery of compounds that inhibit the production or release of reactive oxygen metabolites and/or inflammatory cytokines is described.

### DESCRIPTION OF THE RELATED ART

Reactive oxygen metabolites are often produced by the incomplete reduction of oxygen. The complete reduction of one molecule of O₂ to water is a four-electron process. Oxidative metabolism continually generates partially reduced species of oxygen, which are far more reactive, and hence more toxic than O₂ itself. A one-electron reduction of O₂ yields superoxide ion (O₂⁻); reduction by an additional electron yields hydrogen peroxide (H₂O₂), and reduction by a third electron yields a hydroxyl radical (OH.), and a hydroxide ion. Nitrous oxide (NO), is another interesting reactive oxygen metabolite, produced through an alternative pathway. Hydroxyl radicals in particular are extremely reactive and represent the most active mutagen derived from ionizing radiation. All of these species are generated during the reduction of oxygen and must be converted to less reactive species if the organism is to survive.

Particular cells of the immune system have harnessed the toxic effects of ROMs as an effector mechanism. Professional phagocytes, polymorphonuclear leukocytes (neutrophils, PMN), monocytes, macrophages, and eosinophils function to protect the host in which they reside from infection by seeking out and destroying invading microbes. These phagocytic cells possess a membrane-bound enzyme system that can be activated to produce toxic oxygen radicals in response to a wide variety of stimuli.

The “increased respiration of phagocytosis” (the respiratory burst) was reported and thought to be a result of increased mitochondrial activity providing additional energy for the processes of phagocytosis. It was later shown that a non-mitochondrial enzymatic system produced the increased levels of oxygen metabolites since the respiratory burst continued even in the presence of mitochondrial inhibitors such as cyanide and antimycin A. In 1968, Paul and Sbarra showed clearly that stimulated phagocytes produced hydrogen peroxide and in 1973, Babior and co-workers established that superoxidase was a major product of the superoxidase. (Paul and Sbarra, Biochim Biophys Acta 156(1): 168-78 (1968); Babior, et al., J Clin Invest 52(3): 741-4 (1973). It is now generally accepted that the enzyme is membrane bound, exhibits a preference for NADPH (Kₘ=45 μM) over NADH (Kₘ=450 μM), and converts oxygen to its one electron-reduced product, superoxide.

NADPH+H⁺+2O₂→NADP⁺+2H⁺+2O₂⁻

The hydrogen peroxide arises from subsequent dismutation of the superoxide.

2O₂⁻+2H⁺→H₂O₂+O₂⁻

The enzyme activity is almost undetectable in resting (unstimulated) phagocytes, but increases dramatically upon stimulation. Patients with the rare genetic disorder chronic granulomatous disease (CGD) have a severe predisposition to chronic recurrent infection. The neutrophils from these patients phagocytose normally but the respiratory burst is absent and NADPH oxidase activity (and radical production) is undetectable, indicating that the oxidase and its product, the reactive oxygen metabolites, have an important bactericidal function.

Neutrophils and macrophages produce oxidizing agents to break through the protective coats or other factors that protect phagocytosed bacteria. The large quantities of superoxide, hydrogen peroxide, and hydroxyl ions are all lethal to most bacteria, even when found in very small quantities.

While there are beneficial effects of these oxygen metabolites, it is clear that inappropriate production of oxygen metabolites can result in severely deleterious effects. A number of these deleterious effects manifest themselves in the intraocular tissues of a host. For example, a variety of macular degeneration and retinal damage can be exacerbated by unwanted concentrations of reactive oxygen metabolites. Effective compositions and methods to reduce and minimize the production and release of ROMs in patients suffering from a variety of disparate ocular disorders would be a great boon to medicine and serve to reduce and eliminate a substantial amount of human suffering.

### SUMMARY OF THE INVENTION

Methods and compositions are described for treating intraocular damage caused by trauma, autoimmune disease, degenerative diseases and cellular release of reactive oxygen species or inflammatory cytokines. In one aspect of the invention, a method of treating proliferative diabetic retinopathy is provided. Advantageously, the method includes the identification of a subject presenting the symptoms of proliferative diabetic retinopathy and the administration to at least one of the subject's eyes a pharmaceutically acceptable solution containing an effective concentration of a compound effective to reduce the amount of ROM in an individual. The compound preferably includes a compound effective to inhibit the production or release of enzymatically produced ROM, an ROM scavenger, and combinations thereof.

The compound effective to inhibit the production or release of enzymatically produced ROM may include histamine, histamine phosphate, histamine dihydrochloride, histamine receptor agonists, NADPH oxidase inhibitors, serotonin and serotonin agonists. Alternatively, the compound effective to inhibit the production or release of enzymatically produced ROM may be a scavenger such as catalase, glutathione peroxidase, ascorbate peroxidase, superoxide dismutase, vitamin A, vitamin E, and vitamin C. Optionally, the compound effective to inhibit the production or release of enzymatically produced ROM is a compound that promotes the release of endogenous histamine stores such as IL-3, retinoic acid, 9-cis-retinoic acid, all-trans-retinoic acid, and allergens. Advantageously, the compound is administered intravitreally, topically, or systemically to promote intraocular health and to treat and prevent,intraocular damage caused by ROMs.

In another aspect of the invention, a method of treating preproliferative diabetic retinopathy is provided. The method includes identifying a subject presenting the symptoms or preproliferative diabetic retinopathy; and administering to at least one eye of the subject a pharmaceutically acceptable solution containing an effective concentration of a compound effective to reduce the amount of ROM in an individual. Advantageously, the compound can include a compound effective to inhibit the production or release of enzymatically produced ROM, a ROM scavenger, and combinations thereof. The compound effective to inhibit the production or release of enzymatically produced ROM may be histamine, histamine phosphate, histamine dihydrochloride, histamine receptor agonists, NADPH oxidase inhibitors, serotonin or serotonin agonists. Alternatively, the compound may be a scavenger such as catalase, glutathione peroxidase, ascorbate peroxidase, superoxide dismutase, vitamin A, vitamin E, or vitamin C. In yet another aspect of the invention, the compound effective to inhibit the production or release of enzymatically produced ROM is a compound that promotes the release of endogenous histamine stores such as IL-3, retinoic acid, 9-cis-retinoic acid, all-trans-retinoic acid, and allergens. Advantageously, the compound is administered intravitreally, topically, or systemically.

In still another aspect of the invention, a method of treating proliferative retinopathy is provided. The method includes identifying a subject presenting the symptoms of proliferative retinopathy; and administering to at least one eye of the subject a pharmaceutically acceptable solution containing an effective concentration of a compound effective to reduce the amount of ROM in an individual. Advantageously, the compound can include a compound effective to inhibit the production or release of enzymatically produced ROM, a ROM scavenger, and combinations thereof. The compound effective to inhibit the production or release of enzymatically produced ROM may be histamine, histamine phosphate, histamine dihydrochloride, histamine receptor agonists, NADPH oxidase inhibitors, serotonin or serotonin agonists. Alternatively, the compound may be a scavenger such as catalase, glutathione peroxidase, ascorbate peroxidase, superoxide dismutase, vitamin A, vitamin E, or vitamin C. In yet another aspect of the invention, the compound effective to inhibit the production or release of enzymatically produced ROM is a compound that promotes the release of endogenous histamine stores such as IL-3, retinoic acid, 9-cis-retinoic acid, all-trans-retinoic acid, and allergens. Advantageously, the compound is administered intravitreally, topically, or systemically.

A method of treating age-related macular degeneration is likewise provided, wherein the method includes identifying a subject presenting the symptoms of age-related macular degeneration; and administering to at least one eye of the subject a pharmaceutically acceptable solution containing an effective concentration of a compound effective to reduce the amount of ROM in an individual. Advantageously, the compound can include a compound effective to inhibit the production or release of enzymatically produced ROM, a ROM scavenger, and combinations thereof. The compound effective to inhibit the production or release of enzymatically produced ROM may be histamine, histamine phosphate, histamine dihydrochloride, histamine receptor agonists, NADPH oxidase inhibitors, serotonin or serotonin agonists. Alternatively, the compound may be a scavenger such as catalase, glutathione peroxidase, ascorbate peroxidase, superoxide dismutase, vitamin A, vitamin E, or vitamin C. In yet another aspect of the invention, the compound effective to inhibit the production or release of enzymatically produced ROM is a compound that promotes the release of endogenous histamine stores such as IL-3, retinoic acid, 9-cis-retinoic acid, all-trans-retinoic acid, and allergens. Advantageously, the compound is administered intravitreally, topically, or systemically.

In yet another aspect of the invention, a method of treating retinitis pigmentosa is provided. The method includes identifying a subject presenting the symptoms of retinitis pigmentosa; and administering to at least one eye of the subject a pharmaceutically acceptable solution containing an effective concentration of a compound effective to reduce the amount of ROM in an individual. Advantageously, the compound can include a compound effective to inhibit the production or release of enzymatically produced ROM, a ROM scavenger, and combinations thereof The compound effective to inhibit the production or release of enzymatically produced ROM may be histamine, histamine phosphate, histamine dihydrochloride, histamine receptor agonists, NADPH oxidase inhibitors, serotonin or serotonin agonists. Alternatively, the compound may be a scavenger such as catalase, glutathione peroxidase, ascorbate peroxidase, superoxide dismutase, vitamin A, vitamin E, or vitamin C. In yet another aspect of the invention, the compound effective to inhibit the production or release of enzymatically produced ROM is a compound that promotes the release of endogenous histamine stores such as IL-3, retinoic acid, 9-cis-retinoic acid, all-trans-retinoic acid, and allergens. Advantageously, the compound is administered intravitreally, topically, or systemically.

In another aspect of the invention, a method of treating macular holes is provided. The method includes identifying a subject presenting the symptoms of macular holes; and administering to at least one eye of the subject a pharmaceutically acceptable solution containing an effective concentration of a compound effective to reduce the amount of ROM in an individual. Advantageously, the compound can include a compound effective to inhibit the production or release of enzymatically produced ROM, a ROM scavenger, and combinations thereof. The compound effective to inhibit the production or release of enzymatically produced ROM may be histamine, histamine phosphate, histamine dihydrochloride, histamine receptor agonists, NADPH oxidase inhibitors, serotonin or serotonin agonists. Alternatively, the compound may be a scavenger such as catalase, glutathione peroxidase, ascorbate peroxidase, superoxide dismutase, vitamin A, vitamin E, or vitamin C. In yet another aspect of the invention, the compound effective to inhibit the production or release of enzymatically produced ROM is a compound that promotes the release of endogenous histamine stores such as IL-3, retinoic acid, 9-cis-retinoic acid, all-trans-retinoic acid, and allergens. Advantageously, the compound is administered intravitreally, topically, or systemically.

In still another aspect of the invention, a pharmaceutical composition including a pharmaceutically acceptable ophthalmic solution containing an effective concentration of a compound effective to reduce the amount of ROM in an individual is provided. The ophthalmic solution is optionally formulated for intravitreal, topical, or systemic administration. Advantageously, the compound is a compound effective to inhibit the production or release of enzymatically produced ROM, a ROM scavenger, or combinations thereof. The compound effective to inhibit the production or release of enzymatically produced ROM may include histamine, histamine phosphate, histamine dihydrochloride, histamine receptor agonists, NADPH oxidase inhibitors, serotonin or serotonin agonists. Alternatively, The composition of claim 45, wherein scavenger is selected from the group consisting of catalase, glutathione peroxidase, ascorbate peroxidase, superoxide dismutase, vitamin A, vitamin E, and vitamin C. Optionally, the compound effective to inhibit the production or release of enzymatically produced ROM is a compound that promotes the release of endogenous histamine stores such as IL-3, retinoic acid, 9-cis-retinoic acid, all-trans-retinoic acid, and allergens.

Advantageously, the effective concentration of the compound effective to reduce the amount of ROM in an individual is between about 0.001 to 10% by weight of the ophthalmic solution. In a particularly preferred embodiment, the effective concentration of the compound effective to reduce the amount of ROM in an individual is between about 0.05 and 5% by weight of the ophthalmic solution.

### DETAILED DESCRIPTION OF THE INVENTION

The invention described below relates to compositions and methods for the reduction of reactive oxygen metabolite (ROM) mediated damage in the treatment of intraocular disorders caused by or aggravated by ROMs. The compositions and methods described are useful, for example, for treating certain disorders caused by various disease etiologies including macular degeneration, trauma, and retinal damage.

When injury occurs, whether caused by bacteria, trauma, chemicals, heat, or any other phenomenon, multiple substances that cause dramatic secondary changes in te tissues are released. These secondary changes are called inflammation. Inflammation is characterized by vasodilation of the local blood vessels, creating excess local blood flow, increased permeability of the capillaries with leakage of large quantities of fluid into the interstitial spaces, and other effects.

Soon after the onset of inflammation, neutrophils, macrophages, and other cells invade the inflamed area. Ideally, these cells operate to rid the tissue of infectious or toxic agents. One method these cells use to defend the body from harmful foreign substances includes the production and release of ROMs.

A variety of reactive oxygen metabolites are produced in the monovalent pathway of oxygen reduction. These ROMs are enzymatically produced by phagocytes such as monocytes and polymorphonuclear neutrophils (PMNs) and frequently released in a respiratory burst. Hydrogen peroxide and other ROMs play an important role in a host's immunological defenses. Nevertheless, ROMs produced in excessive amounts or at inappropriate times or locations act to damage a host's cells and tissues, and thus can be detrimental to the host.

Recent work has indicated that intraocular diseases may be caused or exacerbated by ROS. ROS can have direct effects on various cells within the ocular region, leading to apoptosis. Another possible mechanism by which these molecules can damage ocular cells and tissue may be related to the effect ROS have on actuator cells of the immune system. For example, ROS evolved from monocytes and other sources have been shown to effectively suppress the activation and activity of NK cells and T-cells.

The effects of ROM production are many faceted. ROMs are known to cause apoptosis in NK cells. ROMs are also known to cause anergy and apoptosis in T-cells. The mechanisms by which ROMs cause these effects are not fully understood. Nevertheless, some commentators believe that ROMs cause cell death by disrupting cellular membranes and by changing the pH of cellular pathways critical for cell survival.

Additionally, phagocytes that undergo a respiratory burst and produce and release large quantities of ROMs also produce and release secondary cytokines such as tumor necrosis factor-alpha (TNF-α) and interleukin-1 (IL-1). An example of secondary cytokine mediated cell damage is found in the Shwartzman Reaction, where neutrophil mediated cell damage is thought to be activated by TNF and IL-1. Imamura S, et al., “Involvement of tumor necrosis factor-alpha, interleukin-1 beta, interleukin-8, and interleukin-1 receptor antagonist in acute lung injury caused by local Shwartzman reaction” Pathol Int. 47(1): 16-24 (1997). The release of ROMs and cytokines augments the cell damage inflicted by a variety of sources as these potent chemical compounds are disseminated throughout the body. Although released as a defensive measure by the cells of the immune system, the ROMs result in ROM-mediated cell damage and the secondary cytokines cause a rapid deterioration of the patient resulting often in death.

It is one of the surprising discoveries described below that compounds that reduce or inhibit the amount of ROMs and secondary cytokines produced or released by sources within a subject can facilitate the treatment and recovery of individuals suffering from a variety of intraocular disorders. Some of the conditions contemplated as treatable using the described methods and compositions result from a disparate number of etiological causes. Nevertheless, they share a common feature in that their pathological conditions are either caused or exacerbated by enzymatically produced ROM-mediated oxidative damage caused by inappropriate and harmful concentrations of ROMs. For example, one model to explain the efficacy of ROM production and release inhibitors for treating intraocular diseases holds that macrophages and monocytes can contribute to retinal damage caused or linked to new or abherent vessel formation. These cells produce and release ROMs that can damage intraocular tissues. The administration of ROMs production and release inhibitors such as histamine serve to minimize the ROM-mediated damage influenced by the presence of macrophages and monocytes in the intraocular space.

A method of treating and/or preventing intraocular damage caused or exacerbated by ROMs is provided. Thus, the administration of compounds that inhibit the production or release of ROMs, or scavenge ROMs, alone or in combination with other beneficial compounds, offers an effective treatment for a variety of intraocular conditions. In preferred embodiments, various histamine and histamine-related compounds are used to achieve a beneficial reduction or inhibition of enzymatic ROM production and release or the net concentration thereof. In a particularly preferred embodiment, the ROM inhibiting compound is histamine. Importantly, the term “histamine” as used herein incorporates a variety of histamine and histamine related compounds. For example, histamine, the dihydrochloride salt form of histamine (histamine dihydrochloride), histamine diphosphate, other histamine salts, esters, or prodrugs, and histamine receptor agonists are to be included. Also included within the meaning of the term “histamine” are histamine binding mimics and histamine receptor analogs.

The administration of compounds that induce the release of endogenous histamine from an individual's own tissue stores is also included within the scope of the present disclosure. Such compounds include IL-3, retinoids, and allergens. As used herein, the term “histamine” also encompasses compounds which induce the release of endogenous histamine from an individual's own tissue stores. Similarly, other ROM production and release inhibitory compounds such as NADPH oxidase inhibitors like diphenyleneiodonium as well as serotonin, serotonin analogs, and 5HT-receptor agonists are likewise included within the meaning of the term “histamine.”

The compositions and methods disclosed herein also encompass the administration of a variety of ROM scavengers. The term “histamine” as used throughout the specification therefore also includes compounds that scavenge ROM. Known scavengers of ROM include the enzymes catalase, superoxide dismutase (SOD), glutathione peroxidase and ascorbate peroxidase. Additionally, vitamins A, E, and C are known to have scavenger activity. Minerals such as selenium and manganese can also be efficacious in combating ROM-mediated damage. The scope of the methods disclosed herein includes the administration of the compounds listed and those compounds with similar ROM inhibitor activity. The compositions and methods disclosed herein also provide an effective means for preventing and/or inhibiting the release of enzymatically generated ROM in excessive amounts or at inappropriate times or locations.

Formulations

Advantageously, the administration of the ROM production or release inhibiting or scavenging compounds can be by intraocular injection, systemic administration, or topical administration (e.g., eye drops, gels, salves, and the like). However, one of skill in the art will appreciate that other effective methods of administrations are contemplated by the invention. To facilitate administration by injection, a variety of formulations for the application of the compounds described herein are contemplated. The formulations of the described herein facilitate the administration of compounds that inhibit the production or release of ROMs or scavenge ROMs once released. The formulations include an injectable vehicle suitable for the administration of an effective amount of the ROM inhibiting and/or scavenging compounds of the described.

The histamine is present in the pharmaceutical formulations in an amount effective to reduce intraocular damage. The concentration of histamine, or a similarly functioning compound, in the formulations described herein is expressed in terms of percent histamine by weight of the total composition. For example, in one embodiment, histamine is present in an amount between about 0.001 and 10 percent by weight. In another embodiment, histamine is present in an amount between about 0.05 and 5 percent by weight. In still another embodiment, histamine is present in an amount of between about 0.1 and 1 percent by weight.

The formulations described herein comprise histamine and a pharmaceutically acceptable carrier. In a preferred embodiment, the carrier is a sterile, aqueous solution that is buffered with compounds such as phosphate buffers, carbonate buffers and the like. A topical composition is preferably provided as a buffered aqueous solution having a viscosity of from about 1 to 50 centipoise (cps). In another preferred embodiment, the composition is formulated as a viscous liquid having a viscosity of between about 50 and several thousand cps using viscosity-enhancing agents such as, for example propylene glycol, hydroxymethyl cellulose or glycerin.

Other ophthalmic histamine-containing pharmaceutical carriers are also provided, including, for example, gels and ointments. The formulations can also comprise ingredients that regulate the osmolarity of the final formulation, as well as the pH of the formulations.

Alternatively, the histamine containing formulations are adapted for intraocular injection.

For example, the resulting preparations for ocular use are advantageously hypotonic, and have an osmolarity of between about 140 and 280 mOsm/l, and a pH of between about 6.8 and 7.6. The osmolarity of the solutions can be adjusted by means of well known osmolarity adjusting agents such as sodium chloride, potassium chloride and monosaccharides. Alternatively, the resulting preparations can be isotonic, or in another embodiment, the resulting preparations can be hypertonic. The present formulations may also contain other conventional ingredients used in ophthalmic preparations, such as dextrose, preservatives (e.g. Thimerosal™, i.e., sodium ethylmercurithiosalicylate (Sigma; St. Louis, Mo.), benzalkonium chloride), corticosteroids (e.g. prednisone), analgesics (e.g., ibuprofen), antibiotics (e.g., gentamicin, streptomycin), antioxidants (e.g. ascorbic acid, BHA, BHT), demulcents (e.g., glycerin, propylene glycol), and the like. Descriptions of compounds used in standard ophthalmic formulations may be found in, for example, Remington's Pharmaceutical Sciences, latest edition, Mack Publishing Co. Easton, Pa., and in U.S. Pat. Nos. 5,951,971, 5,861,148, and 5,800,807.

The pH of the formulations described herein can be adjusted to the desired value by adding an acid, such as hydrochloric acid, or a base such as sodium hydroxide, until the pH of the formulation falls within the range described above. Such adjustments are preferably made without increasing the ionic strength of the formulation to beyond acceptable levels.

The present histamine-containing compositions are prepared according to conventional techniques by mixing the relative ingredients in appropriate amounts in sterile water, or preparing histamine-containing gels and ointments using gel and ointment preparation techniques well known in the pharmaceutical arts. In preferred embodiments, the formulations are sterilized prior to use.

The ophthalmic formulations described herein are administered to the eyes of a subject, preferably an animal such as a dog, cat, bird, reptile or amphibian, more preferably a mammal, most preferably a human, by any route and through any means where delivery of the histamine content of the formulation to the site of ocular irritation can be achieved. For example, the formulations are administered by spray, by ophthalmic gel, by eye drop, by injection within the eye, or by other methods of administration well known to those of skill in the relevant art. In one embodiment, daily dosages in human therapy of the present ophthalmic formulations are of about 1-2 drops per eye, administered about 1-8 times a day (for instance by means of a standard pharmacopoeia medicinal dropper of 3 mm in external diameter, which when held vertically delivers 20 drops of water of total weight of 0.9-1-1 grams at 25° C.)

Various histamine or histamine-related compounds can be used to achieve a beneficial reduction in the concentration of enzymatically produced ROM. The described invention is also directed to inhibiting ROM production and release.

Typically, the injectable formulations described herein contain the ROM inhibitory or scavenging compounds in a concentration effective to prevent or reduce ROM mediated damage.

The compositions and methods described herein further include administrating a variety of ROM scavengers in conjunction with the ROM production and release inhibiting compounds described above. Known scavengers of ROMs include the enzymes catalase, superoxide dismutase (SOD), glutathione peroxidase and ascorbate peroxidase. Additionally, vitamins A, E, and C are known to have scavenger activity. Minerals such as selenium and manganese can also be efficacious in combating ROM-mediated damage. It is intended that the methods described herein include the administration of the compounds listed and those compounds with similar ROM inhibitor activity.

The concentration of the ROM inhibiting or scavenging described herein can vary in accordance with the other ingredients used in the formulation. In some embodiments, substances such as analgesics are likewise contemplated for inclusion in the compositions described herein. Also, compounds that result in the stimulation of a host's immune system such as cytokines, (for example, IL-1, IL-2, IL-12, IL-15, IFN-α, IFN-β, IFN-γ and the like) may be included in the compositions described herein.

Preferred dosage range can be determined using techniques known to those having ordinary skill in the art. IL-1, IL-2 or IL-12 can be administered in an amount of from about 1,000 to about 300,000 U/kg/day; more preferable, the amount is from about 3,000 to about 100,000 U/kg/day, and even more preferably, the amount is from about 5,000 to about 20,000 U/kg/day.

IFN-alpha, IFN-beta, and IFN-gamma can be administered in an amount of from about 1,000 to about 300,000 U/kg/day; more preferable, the amount is from about 3,000 to about 100,000 U/kg/day, and even more preferably, the amount is from about 10,000 to about 50,000 U/kg/day.

The analgesics, and the immuno-stimulatory compositions can be added singularly to the compositions described herein, or in combination with each other.

Suitable preservatives for use in the formulations described herein include, but are not limited to antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. In addition, combinations or mixtures of these preservatives can be used in the formulations described herein.

Compound Administration

Administration of the compounds described herein is advantageously accomplished through an intraocular injection. Solutions of the active compounds in the form of free acids or pharmaceutically acceptable salts can be administered in water with or without a tenside such as hydroxypropylcellulose. Dispersions making use of glycerol, liquid polyethyleneglycols, or mixtures thereof with oils can likewise be employed for formulating an intraocular delivery system. Additionally, antimicrobial compounds can also be added to the preparation to reduce the incidence of intraocular infection and/or to augment the activity of the histamine-related compound.

Injectable preparations may include sterile water-based solutions or dispersions and powders that can be dissolved or suspended in a sterile medium prior to use. Carriers such as solvents or dispersants containing, e.g., water, ethanolpolyols, vegetable oils and the like can also be added. Coatings such as lecithin and tensides can be used to maintain suitable fluidity of the preparation. Isotonic substances such as sugar or sodium chloride can also be added, as well as products intended to retard absorption of the active ingredients, such as aluminum monostearate and gelatin. One of skill in the art will appreciated that sterile injectable solutions are prepared in the familiar way and filtered before storage and/or administration. Sterile powders can be vacuum-dried or freeze-dried from a solution or suspension.

All substances added to the preparation must be pharmaceutically acceptable and essentially nontoxic in the quantities used. The preparation and formulations that produce a delayed release are also part of the invention. Volumes from 1 to 1000 microliters can be used to inject into a subject's eye.

Controlled release preparations can be achieved by the use of polymers to complex or absorb the histamine. The controlled delivery can be exercised by selecting appropriate macromolecule such as polyesters, polyamino acids, polyvinylpyrrolidone, ethylenevinyl acetate, methylcellulose, carboxymethylcellulose, and protamine sulfate, and the concentration of these macromolecule as well as the methods of incorporation are selected in order to control release of active compound.

Hydrogels, wherein the histamine compound is dissolved in an aqueous constituent to gradually release over time, can be prepared by copolymerization of hydrophilic mono-olefinic monomers such as ethylene glycol methacrylate. Matrix devices, wherein the histamine is dispersed in a matrix of carrier material, can be used.

In another embodiment, the ROM inhibiting compound can be formulated in a pharmaceutically acceptable form for systemic administration at a dosage of approximately 0.2 to 2.0 mg or 3-200 μg/kg. ROM scavenging compounds can also be administered in combination with the ROM production and release inhibitory compounds described above. When the ROM inhibiting or scavenging compound is administered orally, the composition can be formulated as a tablet comprising between 10 mg to 2 grams of active ingredient. A tablet can include 10, 20, 50, 100, 200, 500, 1,000, or 2,000 milligrams of ROM inhibiting or scavenging compound. Preferably, the amount of ROM inhibiting or scavenging compound in a tablet is 100 mg. In some embodiments, the composition includes histamine protectors such as diamine oxidase inhibitors, monoamine oxidase inhibitors and n-methyl transferases.

The treatment can also include periodically boosting patient blood ROM inhibiting or scavenging compound levels by administering 0.2 to 2.0 mg or 3-200 μg/kg of the disclosed compounds injected or ingested 1, 2, or more times per day over a period of one to two weeks at regular intervals, such as daily, bi-weekly, or weekly in order to establish blood levels of ROS inhibiting or scavenging compound at a beneficial concentration such that ROM production and release is inhibited. The treatment is continued until the causes of the patient's underlying disease state is controlled or eliminated.

Administration of each dose of ROM inhibiting or scavenging compound can occur from once a day to up to about four times a day, with twice a day being preferred. Administration can be intravenous, intraocular, intravitreal, oral, transdermal, intranasal, or rectal and can utilize direct hypodermic or other injection or infusion means, or can be mediated by a controlled release mechanism. Any controlled release vehicle or infusion device capable of administering a therapeutically effective amount of the disclosed compounds over a period of time ranging from about 1 to about 90 minutes can be used.

Compounds that scavenge ROM can be administered in an amount of from about 0.1 to about 20 mg/day; more preferably, the amount is from about 0.5 to about 8 mg/day; more preferably, the amount is from about 0.5 to about 8 mg/day; and even more preferably, the amount is from about 1 to about 5 mg/day. Nevertheless, in each case, the dose depends on the activity of the administered compound. The foregoing doses are appropriate for the enzymes listed above that include catalase, superoxide dismutase (SOD), glutathione peroxidase and ascorbate peroxidase. Appropriate doses for any particular host can be readily determined by empirical techniques well known to those of ordinary skill in the art.

Non-enzymatic ROM scavengers can be administered in amounts empirically determined by one of ordinary skill in the art. For example, vitamins A and E can be administered in doses from about 1 to 5000 IU per day. Vitamin C can be administered in doses from about 1 μg to 10 gm per day. Minerals such as selenium and manganese can be administered in amounts from about 1 picogram to 1 milligram per day. These compounds can also be administered as a protective or preventive treatment for ROS mediated disease states.

In addition to histamine, histamine dihydrochloride, histamine phosphate, other histamine salts, esters, congeners, prodrugs, and H₂ receptor agonists, the use of serotonin, 5HT agonists, and compounds which induce release of histamine from the patient's own tissues is also included within the disclosed methods. Retinoic acid, other retinoids such as 9-cisretinoic acid and all-trans-retinoic acid, IL-3 and ingestible allergens are compounds that are known to induce the release of endogenous histamine. These compounds can be administered to the patient by oral, intravenous, intraocular, intravitreal, and other approved routes. The rate of administration should result in a release of endogenous histamine resulting in a blood plasma level of histamine of about 20 nmol/dl.

Administration of each dose of a compound which induces histamine release can occur from once per day to up to about four times a day, with twice per day being preferred. Administration can be oral, intravenous, intraocular, intravitreal, or transdermal, and can incorporate a controlled release mechanism. Any controlled release vehicle capable of administering a therapeutically effective amount of a compound which induces histamine release over a period of time ranging from about one to about thirty minutes can be used. Additionally, the compounds, compositions, and formulations described herein can be administered quantum sufficiat.

The following examples teach the methods of the present invention and the use of the disclosed ROM production and release inhibiting compounds. These examples are illustrative only and are not intended to limit the scope of the present invention. The treatment methods described below can be optimized using empirical techniques well known to those of ordinary skill in the art. Moreover, artisans of ordinary skill would be able to use the teachings described in the following examples to practice the full scope of the present invention.

### EXAMPLE 1

Histamine Treatment of Proliferative Diabetic Retinopathy (PDR)

Diabetic retinopathy is the leading cause of blindness in working age Americans. The incidence of retinopathy increases with the time of the disease state, from a level of about 50% manifestation in diabetics with the disease for 7 years to approximately 90% of those with the disease for more than 20 years. It is estimated that PDR affects an estimated 700,000 Americans.

The retinovascular consequences of diabetes essentially consist, in part, of microvascular leakage and capillary nonperfusion resulting from chronic hyperglycemia. Microvascular leakage may in turn result in retinal edema, lipid exudates and intraretinal hemorrhages. Capillary nonperfusion results in the formation of intraretinal microvascular abnormalities (IRMA). These abnormalities include the development of arteriovenous shunts formed to perfuse retinal regions deprived of vascularization by diabetes-mediated arteriole degeneration.

Expression of vascular endothelial growth factor from an hypoxic retina in areas of capillary nonperfusion is thought to result in the development of extraretinal neovascularization. Such neovascularization and its associated fibrous components may spontaneously involute or be complicated by vitreous hemorrhage or traction retinal detachment. Neovascularization may be easily seen on fluorescein angiogram by the profuse leakage of dye from these new vessels since they lack the tight endothelial junctions of the retinal vasculature. Impaired axoplasmic flow in areas of retinal hypoxia result in cotton wool spots.

Proliferative diabetic retinopathy (PDR) requires careful screening of diabetics for early identification and treatment since PDR remains largely asymptomatic in the early stages. Proliferative diabetic retinopathy can be classified into three subgroups: (1) nonproliferative retinopathy; (2) preproliferative retinopathy; and (3) proliferative retinopathy. Each classification has certain morphological characteristics. Features of nonproliferative retinopathy include capillary microangiopathy (microvascular obstructions and permeability changes, nonperfusion of capillaries, retinal capillary microaneurysms, basement membrane thickening and internal microvascular abnormalities (IRMA); intraretinal hemorrhages; exudates; and macular changes. Preproliferative retinopathy is indicated by any or all of the changes described for nonproliferative retinopathy and the following additional symptoms: significant venous beading, cotton-wool exudates, extensive IRMA and extensive retinal ischemia. Proliferative retinopathy is indicated by the presence of extraretinal neovascularization and fibrous tissue proliferation, vitreous alterations and hemorrhage, macular disease, and retinal detachment.

The creation of fibrovascular tissue is an especially important complication of PDR since it often will lead to retinal damage mediated by the vitreous. The fibrovascular tissue may form preretinal membranes that create dense adhesions with the posterior hyaloid membrane. These adhesions are responsible for transmitting the forces of vitreous traction to the retina, which may result in retinal detachments.

The vitreous base is normally firmly attached to the adjacent retina and to the outer circumference of the optic nerve head, known as the ring of Martegiani. The attachmet of the vitreous to the retina in all other sites between the ring of Martegiani and the vitreous base is much less firm. Neovascularization from the retina leads to the formation of vascular strands extending into the vitreous from the nerve head or elsewhere in the fundus. Contraction of these strands may cause partial or complete retinal detachment.

Retinal detachment at the macula is a major complication of PDR. Most retinal detachments resulting from PDR begin as tractional detachments without holes, but they may become rhegmatogenous by the formation of retinal holes at some later point in the disease. The tractional detachments are caused by abnormal vitreoretinal adhesions or vitreal traction with subsequent shrinkage of the fibrous bands and elevation of the retina.

The methods described can be used to treat PDR in the preproliferative and proliferative states using intravitreal injections of histamine or other suitable ROM inhibiting or scavenging compound. Without being limited to a particular mechanism, it is believed that the effect of intravitreal histamine injection is to inhibit retinal damage caused or exacerbated by ROMs. It is further contemplated that the histamine described herein may be performed alone or in combination with other treatments of PDR.

As a preliminary step a patient is identified as suffering from PDR. A volume of approximately 100 μl of a 2% histamine-containing solution is injected intraocularly into the effected eye or eyes. The patient is monitored thereafter. The treatment is repeated every two weeks. A reduction in symptoms associated with PDR is observed following the administration of histamine.

### EXAMPLE 2

Treatment of Preproliferative Diabetic Retinopathy

A diabetic patient manifesting preproliferative diabetic retinopathy is treated for this complication of diabetes mellitus through the intravitreal injection of a histamine compound. The purpose of this treatment is to reduce or prevent the development of proliferative diabetic retinopathy manifested by extraretinal neovascularization and fibrous tissue proliferation, vitreous alterations and hemorrhage, macular disease, and retinal detachment.

Once a patient has been diagnosed with diabetes, increased ophthalmic surveillance is performed, given the high percentage of individuals suffering from this disease later developing proliferative diabetic retinopathy (PDR). This increased surveillance should include periodic retinal examinations and fluorescein angiograms to monitor the extent of venous beading, IRMA, and retinal ischemia.

When preproliferative diabetic retinopathy begins reaching the proliferative stage, treatment with an ROM inhibitor or scavenger is commenced. This stage is defined as the presence of venous beading in 2 or more quadrants, IRMA in one or more quadrants, and/or microaneurysm and dot hemorrhages in all quadrants. Once these indicia are present, the administration of a ROM inhibitor or scavenger is initiated.

The patient receives a full ophthalmic examination to establish a baseline of ocular health. The ophthalmic examination includes indirect ophthalmoscopy, slit-lamp biomicroscopy, peripheral retinal examination, intraocular pressure measurements, visual acuity (unaided and best corrected) symptomatology, fundus photography, fluorescein angiography, electroretinography and A-scan measurements.

Following the preliminary examination, an intravitreal injection of histamine diphosphate is given to the patient's affected eye. If both eyes are affected, they may be treated separately. The eye to be treated is injected intravitreally with a histamine ophthalmic solution containing 1% histamine diphosphate to prevent or reduce ROM mediated intraocular damage.

After treatment, the patient's eyes are examined on days one (1), two (2), seven (7), fifteen (15), thirty (30) and sixty (60). On each examination day, the patient is monitored. Additionally, the patient is monitored for posterior vitreous detachments using indirect ophthalmoscopy with scleral depression. Finally, the extent of PDR presented by the patient is continuously monitored through periodic retinal examinations and fluorescein angiograms to monitor the extent of venous beading, IRMA, and retinal ischemia.

The administration of histamine diphosphate results in the reduction in the development of proliferative diabetic retinopathy as compared to an untreated individual.

### EXAMPLE 3

Treatment of Proliferative Retinopathy

A diabetic patient manifesting proliferative diabetic retinopathy is treated by the administration of histamine dihydrochloride, which is formulated as an ophthalmic gel. The purpose of this treatment is to reduce the extent of proliferative diabetic retinopathy, to prevent further manifestations of the disease after removal of any extraretinal neovascularized tissue, and to reduce the likelihood of retinal detachment.

A patient presenting proliferative diabetic retinopathy receives the histamine treatment described herein in combination with surgical treatment of the neovascularized tissue. The proliferation usually begins with the formation of new vessels with very little fibrous tissue component. New vessels arise from primitive mesenchymal elements that differentiate into vascular endothelial cells. The newly formed vascular channels then undergo fibrous metaplasia; that is, the angioblastic buds are transformed into fibrous tissue.

The new vessels leak fluorescein, so the presence of proliferation is especially noticeable during angiography. The new vessels and fibrous tissue break through the internal limiting membrane and arborize at the interface between the internal limiting membrane and the posterior hyaloid membrane. The fibrovascular tissue may form preretinal membranes that create dense adhesions with the posterior hyaloid membrane. These adhesions are extremely important because they are responsible for transmitting the forces of vitreous traction to the retina during the later stage of vitreous shrinkage.

The proliferative stage of PDR is defined as the presence of three or more of the following characteristics: new vessels, new vessels on or within one disc diameter of the optic nerve, severe new vessels (as defined by one-third disc area neovascularization at the optic nerve or one-half disc area neovascularization at the optic nerve or one-half disc area neovascularization elsewhere), and preretinal or vitreous hemorrhage.

Once diagnosed as entering the proliferative stage, the patient receives a full ophthalmic examination to establish a baseline of ocular health. The ophthalmic examination includes indirect ophthalmoscopy, slit-lamp biomicroscopy, peripheral retinal examination, intraocular pressure measurements, visual acuity (unaided and best corrected visual acuity) symptomatology, fundus photography, fluorescein angiography, electroretinography and A-scan measurements.

Following the preliminary examination, an ophthalmic gel comprising histamine dihydrochloride is administered to patient's affected eye. If both eyes are affected, the eyes may be treated separately. The eye is treated with the ophthalmic gel comprising histamine dihydrochloride to promote a reduction of ROM levels. The eye to be treated is administered an ophthalmic gel containing 0.5% histamine dihydrochloride to prevent or reduce ROM mediate intraocular damage. In addition, the neovascularized tissue is also treated directly to minimize subsequent damage to the retina using panretinal photo coagulation.

Panretinal photocoagulation (PRP) may be used to treat patients presenting PDR in conjunction with the histamine treatment. Panretinal photocoagulation is a form of laser photocoagulation. Currently lasers such as the argon green (614 nm), argon blue-green (488 and 514 nm), krypton red (647 nm), tunable dye, diode and xenon arc lasers, are used for retinal surgery. Laser energy is absorbed predominantly by tissues containing pigment (melanin, xanthophyll, or hemoglobin) producing thermal effects on adjacent structures. Krypton red lasers are the preferred method of treatment, as they are better able to penetrate nuclear sclerotic cataracts and vitreous hemorrhage than the argon lasers, which require more energy to produce equal levels of penetration.

The parameters used during laser retinal surgery may be modified depending on the goal of the photocoagulation. At lower power setting, using longer durations of treatment and producing larger spot sizes, the laser has a coagulative effect on small vessels. Focal laser photocoagulation is used in diabetes to stop leakage of microaneurysms. The laser spot is place directly over the microaneurysm to achieve a slight whitening and closure of the aneurysm. When applied as a grid over an edematous area of retina, the laser may reduce microvascular leakage. At higher energy levels, laser ablation of tissue is possible. Panretinal photocoagulation is thought to be effective by destroying retinal tissue, reducing the amount of ischemic tissue in the eye. Confluent laser spots may be used over a neovascular membrane to obliterate the abnormal vessels.

It should be understood that the described methods do not require a particular order of treatment. In one embodiment, the patient is first treated with histamine and then laser treatment. In another embodiment the patient is first undergoes laser treatment followed by one or more histamine treatments.

After treatment, the patients' eyes are examined on days one (1), two (2), seven (7), fifteen (15), thirty (30) and sixty (60). On each examination day, the patient is monitored. Additionally, the patient is monitored for posterior vitreous detachments using indirect ophthalmoscopy with scleral depression. Finally, the extent of PDR presented by the patient is continuously monitored through periodic retinal examinations and fluorescein angiograms to monitor the extent of venous beading, IRMA, retinal ischemia, neovascularization, and vitreal hemorrhage. Evidence of new neopolymerization would warrant a repeat treatment of the patient as described above.

A reduction in the development of posterior vitreous detachments is observed in patients treated with ophthalmic gel containing histamine dihydrochloride as compared with patients who received no histamine.

### EXAMPLE 4

Histamine Treatment of Age-Related Macular Degeneration

The described methods have utility in the treatment of age-related macular degeneration (AMD). Age-related macular degeneration consists of a gradual, often bilateral decrease of vision. It is the most common cause of legal blindness in adults. It is probably caused by aging and vascular disease in the choriocapillaries or the afferent retinal vessels. There are basically two morphologic types of AMD: “dry” and “wet”.

The underlying abnormality of AMD is the development of involutional changes at the level of Bruch's membrane and the retinal pigment epithelium (RPE). The hallmark lesion of such changes is the druse. Clinically, drusen (the plural form of druse) appear as small, yellow-white deposits at the level of the RPE. Drusen may be categorized as hard, soft or basal laminar drusen.

The described methods are directed, in part, to both to the treatment and prevention of wet and dry forms of AMD. In the wet form the disease, the condition is thought to affect the choriocapillaries. The choriocapillaries are a component of the choroid, which serves to vascularize the globe. The choriocapillaries consists of a rich capillary network that supply-most of the nutrition for the pigment epithelium and outer layers of the retina. Damage to the choriocapillaries is thought to result ultimately in neovascular complications, a cause of macular degeneration.

In the dry form, nondisciform macular degeneration results from a partial or total obliteration of the underlying choriocapillaries. Ophthalmoscopically, degeneration of the retinal pigment epithelium and hole formation may be observed. Also, subpigment epithelial deposits of material such as calcium chelates and others may be observed. In dry ADM, secondary retinal changes generally occur gradually, resulting in the gradual loss of visual acuity. Nevertheless, in some percentage of patients, a severe loss of vision results.

The described compositions and methods have utility in treating dry ADM and preventing macular degeneration reduction of intraocular ROM concentrations caused by infiltrating phagocytes by administering a compound which inhibits or scavenges ROMs. It is believed that the reduction of intraocular ROM concentrations would reduce macular degeneration.

Wet ADM most frequently results from choriocapillary insufficiency, leading to subsequent subpigment epithelial neovascularization. Neovascularization also is thought to occur as an adaptation of retinal vascularization to inadequate oxygenation as a result of vesicular damage. Neovascularization may also cause several other disorders such as detachment of the pigment epithelium and sensory retina. Typically the disease usually begins after 60 years of age, manifesting in both sexes equally and in patients presenting the disease bilaterally.

Perhaps the most important complication of age-related macular degeneration (AMD) is the development of defects in Bruch's membranes of the globe through which new vessels grow. This epithelial neovascularization may result in the production of exudative deposits in and under the retina. The neovascularization may also lead to hemorrhage into the vitreous, which may lead to degeneration of the retina's rods and cones and cystoid macular edema (discussed below). A macular hole may form which results in irreversible visual loss.

Although affecting only 10% of patients with AMD, neovascular complications of AMD account for the overwhelming majority of cases of severe visual loss. Risk factors include increasing age, soft drusen, nongeographic atrophy, family history, hyperopia, and retinal pigment epithelial detachments. Symptoms of choroidal neovascularization in AMD include metamorphopsia, paracentral scotomas or diminished central vision. Ophthalmoscopic findings include subretinal fluid, blood, exudates, RPE detachment, cystic retinal changes, or the presence of grayish green subretinal neovascular membrane. Fluorescein angiography is often an effective method of diagnosis. During this diagnostic procedure, progressive pooling of the dye in the subretinal space, seen as blurring of the boundaries of the lesion or leakage from undetermined sources are indicators of the disease. Other components of choroidal neovascular membranes as delineated by fluorescein angiography include elevated blocked fluorescence, flat blocked fluorescence, blood, and disciform scar.

The present understanding of neovascular AMD suggests that classic choroidal neovascularization is the lesion component most strongly associated with rapid visual deterioration. Accordingly, treatment of AMD must encompass all neovascular and fibrovascular components of the lesion. At present, treatment is only indicated when classic neovascularization has boundaries that are well demarcated, and photocoagulation has been shown to be beneficial.

In eyes with extrafoveal choroidal neovascularization (>=200 microns from the foveal center), argon laser photocoagulation diminished the incidence of severe visual loss at 5 years from 64% to 46%. Recurrent neovascularization developed in one-half of laser-treated eyes, usually in the first year after treatment. Recurrent neovascularization was invariably associated with the development of severe visual loss.

In eyes with juxtafoveal choroidal neovascularization (1 to 199 microns from the foveal center), krypton laser photocoagulation diminished the incidence of severe visual loss from 45% to 31% at 1 year, although the difference between untreated and treated groups was less marked at 5 years.

Laser treatment remains an essential therapeutic method for the treatment of AMD, however, the described methods would augment the laser treatment by reducing the reoccurrence of neovascularization and its attendant ROM mediate damage caused by the cells responsible for neovascularization.

Following the preliminary examination, eye drops formulated with retinoic acid are administered to patient's affected eye. If both eyes are affected, they may be treated separately. Drops of a retinoic acid ophthalmic solution are administered to promote a reduction of ROM levels. The eye to be treated is administered an ophthalmic solution containing 0.1% retinoic acid formulated as an eye drop to prevent or reduce ROM mediate intraocular damage. A reduction in choroidal neovascularization is observed in eyes treated with retinoic acid as compared with untreated eyes.

### EXAMPLE 5

Treatment of Age-Related Macular Degeneration

A patient manifesting age-related macular degeneration is treated with an intravitreal injection of a scavenger of ROM, namely superoxide dismutase. The purpose of this treatment is to reduce or prevent the development of neovascularization, macular disease, and retinal damage mediated by ROM production and release, and inflammation caused by cellular infiltrates.

Once a patient reaches the age of 60, increased ophthalmic surveillance is performed to detect the presence of ADM. This increased surveillance should include periodic retinal examinations and fluorescein angiograms to monitor for the presence of subretinal fluid, blood, exudates, RPE detachment, cystic retinal changes, or the presence of grayish green subretinal neovascular membrane.

When ADM is diagnosed, a regime of histamine treatment is commenced coupled with or without other treatments such as photocoagulation. As the first step of treatment, the patient receives a full ophthalmic examination to establish a baseline of ocular health. The ophthalmic examination includes indirect ophthalmoscopy, slit-lamp biomicroscopy, peripheral retinal examination, intraocular pressure measurements, visual acuity (unaided and best corrected) symptomatology, fundus photography, fluorescein angiography, electroretinography and A-scan measurements.

Following the preliminary examination, an intravitreal injection of superoxide dismutase is given to the patient's affected eye manifesting ADM. If both eyes are affected, they may be treated separately. The eye to be treated is injected intravitreally with an ophthalmic solution containing 0.75% superoxide dismutase to prevent or reduce ROM mediate intraocular damage.

Laser photocoagulation treatment of the histamine injected eyes may be required. The laser treatment protocol described in Example 5 and 6 should be followed when treating AMD. In an alternative embodiment, photocoagulation treatment occurs before utilization of these described treatment.

After treatment, the patients' eyes are examined on days one (1), two (2), seven (7), fifteen (15), thirty (30) and sixty (60). Because of the possibility of reoccurrence, the patient should return for periodic examinations on a monthly basis thereafter. On each examination day, the patient is monitored for posterior vitreous detachments using indirect ophthalmoscopy with scleral depression. Finally, the extent of ADM presented by the patient is continuously monitored through periodic retinal examinations and fluorescein angiograms to monitor for the presence of subretinal fluid, blood, exudates, RPE detachment, cystic retinal changes, or the presence of grayish green subretinal neovascular membrane. Additional superoxide dismutase and/or laser treatments may be required if indicia of reoccurring neovascularization are observed. An improvement in ocular health is observed in the eyes of patients administered superoxide dismutase as compared to untreated eyes.

The following Example demonstrates the efficacy of the described methods, even without the use of photocoagulation.

### EXAMPLE 6

Histamine Treatment of Retinitis Pigmentosa

Retinitis pigmentosa (RP) is the name given to a group of heritable disorders of progressive retinal degeneration characterized by bilateral nyctalopia constricted visual fields and abnormality of the electroretinogram. Early symptoms include difficulty with dark adaptation and midperipheral visual field loss. As the disease progresses, visual field loss advances, typically leaving a small central field of vision until eventually even central vision is affected. Central acuity may also be affected earlier in the course of disease either by cystoid macular edema, macular atrophy, or by the development of a posterior subcapsular cataract. RP represents a varied group of diseases whose common thread is the abnormal production of at least one protein in photoreceptor outer segments critical to light transduction.

One clinical result of RP is the destabilization of the blood-retinal barrier of the perifoveal capillaries and the optic nerve head. This destabilization results in leakage of fluorescein dye observed by angiography. In addition to leakage, accumulation of fluid as microcysts in the outer plexiform layer may occur and be observed. These fluid-filled cysts may eventually burst, resulting in damage to the retinal layer. The described methods and compositions can be used to treat RP related damage to the retina by reducing ROM mediated damage.

Following the preliminary examination, histamine is topically administered in the form of a salve to a patient's affected eyes. If both eyes are affected, they may be treated separately. A salve comprising 0.05% by weight of an NADPH oxidase inhibitor is topically administered to the affected eye or eyes to promote a reduction of ROM levels, thereby preventing or reducing ROM mediate intraocular damage. An amelioration of symptoms associated with AMD is observed in the eyes of patients who are administered a NADPH oxidase inhibitor as compared to untreated eyes.

### EXAMPLE 7

Histamine Treatment of Macular Holes

A rupture or bursting open of the macula is known as a macular hole. Interestingly, this condition usually occurs in women in their sixth through eighth decades, or after trauma such as lightening injury, solar injury, scleral buckling, or in staphylomatous eyes. Symptoms include metamorphopsia and diminished visual acuity.

Macular hole formation is thought to result from tangential traction across the retinal surface induced by the posterior cortical vitreous with involvement of fluid movement within a posterior vitreous syneresis cavity. The posterior vitreous syneresis cavity is present in the vast majority of patients presenting macular holes. It is thought that as the posterior vitreal gel retreats from the retinal surface, the resulting gap between the two surfaces creates an area wherein movement of the vitreous humor may negatively interact with the retinal surface. The tangential movement of the vitreous humor within the space of the posterior vitreous syneresis cavity is thought to promote tears of the retinal membrane, resulting in the creation of macular holes.

The described methods contemplate the use of histamine to reduce ROM levels and so as to eliminate the conditions which result in macular hole formation. Following the preliminary examination, an intravitreal injection of histamine dihydrochloride is given to patient's affected eye. If both eyes are affected, they may be treated separately. The eye is injected with the histamine ophthalmic solution intravitreally to promote a reduction of ROM levels. The eye to be treated is injected intravitreally with 200 μl of a histamine ophthalmic solution containing 5% histamine dihydrochloride to prevent or reduce ROM mediate intraocular damage.

A reduction in the incidence of macular hole formation is observed in eyes treated with histamine as compared with untreated eyes.

### EXAMPLE 8

Treatment of Macular Holes

A patient presenting the early signs of macular hole formation is treated with an intravitreal injection of histamine. The patient to be treated presents any number of the various signs of premacular hole formation. These signs include loss of the foveal depression associated with a yellow foveal spot or ring. The fovea has begun to thin in the region of hole formation and the lesion may obtain a reddish appearance. Fluorescein angiography at this stage may appear normal or show faint hyperfluorescence. The appearance of an eccentric full thickness dehiscence denotes an advanced early stage of the disease. Upon observance of these symptoms histamine treatment is commenced.

The histamine treatment described herein is commenced when the formation of a macular hole is diagnosed. The patient receives a full ophthalmic examination to establish a baseline of ocular health. The ophthalmic examination included indirect ophthalmoscopy, slit-lamp biomicroscopy, peripheral retinal examination, intraocular pressure measurements, visual acuity (unaided and best corrected) symptomatology, fundus photography, fluorescein angiography, electroretinography and A-scan measurements.

Following the preliminary examination, an intravitreal injection of histamine is given to patient's affected eye. If both eyes are affected, they may be treated separately. The eye is injected with the histamine ophthalmic solution intravitreally to promote a reduction of ROM levels. The eye to be treated is injected intravitreally with 100 μl of a histamine ophthalmic solution containing 1% of a histamine receptor analog to prevent or reduce ROM mediate intraocular damage.

After treatment, the patients' eyes are examined on days one (1), two (2), seven (7), fifteen (15), thirty (30) and sixty (60). On each examination day, the patient's treated eyes are monitored. Fluorescein angiography, considered a particularly effect method of monitoring the course of the treatment, is also performed. Additionally, the patient is monitored for posterior vitreous detachments using indirect ophthalmoscopy with scleral depression.

A reduction in the number and severity of macular holes is observed in eyes injected intravitreally with a histamine receptor analog as compared to untreated eyes.

1. A method of treating proliferative diabetic retinopathy, comprising:
identifying a subject presenting the symptoms of proliferative diabetic retinopathy; and
administering to at least one eye of said subject a pharmaceutically acceptable solution containing an effective concentration of a compound effective to reduce the amount of ROM in an individual.

2. The method of claim 1, wherein said compound is selected from the group consisting of a compound effective to inhibit the production or release of enzymatically produced ROM, an ROM scavenger, and combinations thereof.

3. The method of claim 2, wherein said compound effective to inhibit the production or release of enzymatically produced ROM is selected from the group consisting of histamine, histamine phosphate, histamine dihydrochloride, histamine receptor agonists, NADPH oxidase inhibitors, serotonin and serotonin agonists.

4. The method of claim 2, wherein said ROM scavenger is selected from the group consisting of catalase, glutathione peroxidase, ascorbate peroxidase, superoxide dismutase, vitamin A, vitamin E, and vitamin C.

5. The method of claim 2, wherein said compound effective to inhibit the production or release of enzymatically produced ROM is a compound that promotes the release of endogenous histamine stores.

6. The method of claim 5, wherein said endogenous histamine releasing compound is selected from the group consisting of IL-3, retinoic acid, 9-cis-retinoic acid, all-trans-retinoic acid, and allergens.

7. The method of claim 1, wherein said compound is administered intravitreally, topically, or systemically.

8. A method of treating preproliferative diabetic retinopathy, comprising:
identifying a subject presenting the symptoms or preproliferative diabetic retinopathy; and
administering to at least one eye of said subject a pharmaceutically acceptable solution containing an effective concentration of a compound effective to reduce the amount of ROM in an individual.

9. The method of claim 8, wherein said compound is selected from the group consisting of a compound effective to inhibit the production or release of enzymatically produced ROM, an ROM scavenger, and combinations thereof.

10. The method of claim 9, wherein said compound effective to inhibit the production or release of enzymatically produced ROM is selected from the group consisting of histamine, histamine phosphate, histamine dihydrochloride, histamine receptor agonists, NADPH oxidase inhibitors, serotonin and serotonin agonists.

11. The method of claim 9, wherein said ROM scavenger is selected from the group consisting of catalase, glutathione peroxidase, ascorbate peroxidase, superoxide dismutase, vitamin A, vitamin E, and vitamin C.

12. The method of claim 9, wherein said compound effective to inhibit the production or release of enzymatically produced ROM is a compound that promotes the release of endogenous histamine stores.

13. The method of claim 12, wherein said endogenous histamine releasing compound is selected from the group consisting of IL-3, retinoic acid, 9-cis-retinoic acid, all-trans-retinoic acid, and allergens.

14. The method of claim 8, wherein said compound is administered intravitreally, topically, or systemically.

15. A method of treating proliferative retinopathy, comprising:
identifying a subject presenting the symptoms of proliferative retinopathy; and
administering to at least one eye of said subject a pharmaceutically acceptable solution containing an effective concentration of a compound effective to reduce the amount of ROM in an individual.

16. The method of claim 15, wherein said compound is selected from the group consisting of a compound effective to inhibit the production or release of enzymatically produced ROM, an ROM scavenger, and combinations thereof.

17. The method of claim 16, wherein said compound effective to inhibit the production or release of enzymatically produced ROM is selected from the group consisting of histamine, histamine phosphate, histamine dihydrochloride, histamine receptor agonists, NADPH oxidase inhibitors, serotonin and serotonin agonists.

18. The method of claim 16, wherein said ROM scavenger is selected from the group consisting of catalase, glutathione peroxidase, ascorbate peroxidase, superoxide dismutase, vitamin A, vitamin E, and vitamin C.

19. The method of claim 16, wherein said compound effective to inhibit the production or release of enzymatically produced ROM is a compound that promotes the release of endogenous histamine stores.

20. The method of claim 19, wherein said endogenous histamine releasing compound is selected from the group consisting of IL-3, retinoic acid, 9-cis-retinoic acid, all-trans-retinoic acid, and allergens.
